(19) United States
(12) Patent Application Publication
(10) Pub. No.: US 2005/0005932 A1
(43) Pub. Date: Jan. 13, 2005

(54) Integrated anesthesia machine

(76) Inventor: Mitchell F. Berman, Hastings-on-Hudson, NY (US)

Correspondence Address: Leslie Gladstone Restaino, Esq.;Brown Raysman Millstein Felder & Steiner LLP, 163 Madison Avenue,, P.O. Box 1989, Morristown, NJ, 07962-1989, US

(21) Appl. No.: 10/804,250

(22) Filed: Mar. 19, 2004

Related U.S. Application Data
(60) Provisional application No. 60/456,135, filed on Mar. 19, 2003.

(51) Int. Cl. A61M 15/00; A61M 16/10

(52) U.S. Cl. 128/203.12

(57) ABSTRACT

The present invention provides an integrated anesthesia machine with at least one display adjustably integrated into to the main body of the machine so as not to protrude out of the frame and thereby not obstruct the operator's view of the operation site. The anesthesia machine generally includes a machine frame appropriately equipped for use in administering anesthesia to subjects during a surgical intervention at least one display, such as a flat panel display, for patient monitoring that is adjustably mounted to the machine frame.

### RELATED APPLICATION

This application claims the benefit of U.S. Provisional Application No. 60/456,135.

### BACKGROUND OF THE INVENTION

The present invention relates to medical devices. More particularly, the present invention relates to integrated anesthesia machines.

Anesthesia machines are made available by a number of manufacturers, such as Datex-Ohmeda, which are generally used during surgical interventions to deliver anesthesia to a subject. The machines may be combined with a variety of monitoring systems for monitoring anesthesia delivered, patient cardiac vital signs, etc. Each monitoring system, however, requires a separate display screen. The display screens are typically fixedly mounted onto the anesthesia machine or mounted to hang of the side of the machine. These methods of mounting the displays are problematic insofar as the fixed mounts do not allow for adjustment and the hanging mounts protrude from the device, which get in the way of other equipment and obscure the operating field. Additionally, the anesthesia machines do not provide an operating room compatible keyboard for entry of patient data.

Accordingly, there is a need for anesthesia machines with displays adjustably mounted into the main area of the anesthesia machine directly in front of the operator. There is also a need for anesthesia machines with operating room compatible keyboards.

### SUMMARY OF THE INVENTION

In one aspect of the invention, an anesthesia machine is provided that includes an anesthesia machine frame and at least one display adjustably attached to the frame. The display is mounted in the main body of the anesthesia machine so as not to protrude from the frame. The display is preferably adjustably attached to the frame to move in at least one direction selected from the group consisting of: in an in-out direction with respect to the frame, in a left-right direction with respect to the frame, and in an up-down direction with respect to the frame. The display may also be adjustably attached to the frame to allow the display to tilt in at least one direction selected from the group consisting of: in a direction left to right with respect to the frame and in an up-down direction with respect to the frame.

In another embodiment, the display is adjustable mounted to the frame with a mounting arm having one end adjustably attached to the display and another end adjustably attached to the frame with a horizontal sliding track arranged to provide movement in at least one of: fore and aft, and left and right movement. In another embodiment, the anesthesia machine includes an operating room compatible keyboard, such as one that is waterproof. The keyboard is preferably adjustably attached to the anesthesia machine.

In one embodiment, the display is adopted for patient monitoring. In another embodiment, the anesthesia machine includes a display that provides an interface for automated record keeping. The record keeping interface may be provided with at least one computing device with programs associated therewith that when executed are capable of capturing at least one of patient preoperative data and operative data.

Additional aspects of the present invention will be apparent in view of the description which follows.

### BRIEF DESCRIPTION OF THE FIGURES

FIG. 1 is a front view of an anesthesia machine with a plurality of displays adjustably mounted thereto in the main body of the machine according to one embodiment of the invention.

FIG. 2 is a rear view of the anesthesia machine showing two displays mounted to the main body of the machine according to one embodiment of the invention.

FIG. 3 is a view of the anesthesia machines with an operating room compatible keyboard adjustably mounted thereto according to one embodiment of the invention

FIG. 4 is a rear view of the anesthesia machine according to one embodiment of the invention.

FIG. 5 is a side view showing the mounting details for the display according to one embodiment of the invention.

### DETAILED DESCRIPTION OF THE INVENTION

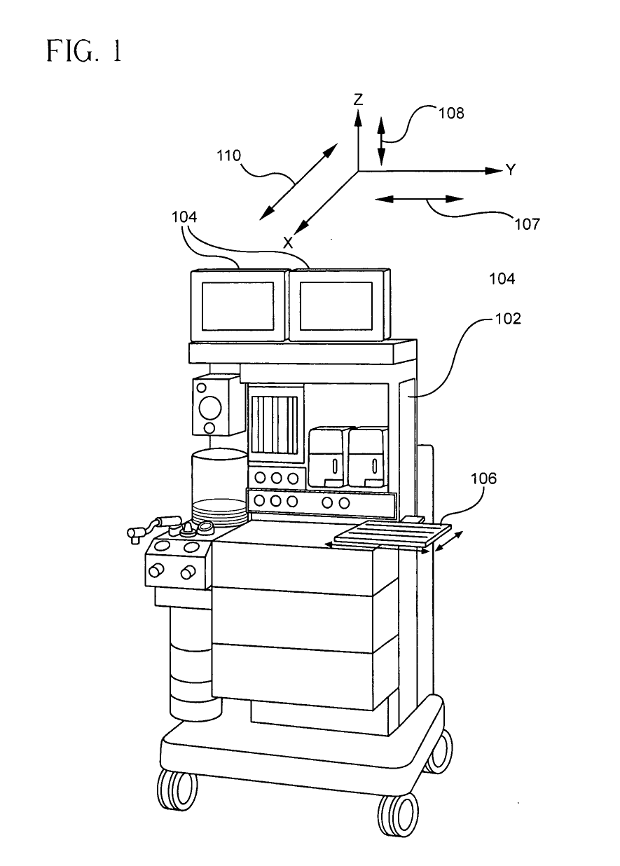

In the operating room environment, it is essential that operator displays be integrated into the main workspace of the anesthesia machines. The present invention generally provides an anesthesia machines with at least one display adjustably integrated into to the main body of the machine so as not to protrude out of the frame and thereby not obstruct the operator's view of the operation site. Referring to FIG. 1, an anesthesia machine according to one embodiment of this invention includes a machine frame 102 appropriately equipped for use in administering anesthesia to patients during a surgical intervention. The anesthesia machine also includes at least one display 104, such as a flat panel display, for patient monitoring that is adjustably mounted to the machine frame 102. The displays 104 are preferably mounted to move in at least one degree of freedom with respect to the frame 102. For instance, the display 104 may be adopted to be adjusted in a X-X direction 110 so as to allow a user to move the displays into and out of the frame 102, in a Y-Y direction 107 to allow a user to move the displays in a left to right direction, and in a Z-Z direction 108 allowing the user to move the display in an up and down direction. In one embodiment, the displays are mounted to allow for left and right, and up and down tilting of the face.

In another embodiment, the machine includes a display 104 for displaying automated record keeping graphic user interfaces associated with record keeping programs. The record keeping programs are generally used to obtain/capture patient pre-operative and operative data, either from the user or automatically from data capture devices designed to monitor patient operative data, such as vital signs, etc. An operating room compatible keyboard 106 may also be operatively connected to the anesthesia machine or the monitoring systems included therewith. The operating room keyboard 106 is preferably waterproof and thus washable for repeated operating room use. In one embodiment, the keyboard 106 is operatively connected to a computing device with patient record keeping programs or software associated therewith. The keyboard 106 is preferably adjustably mounted to the frame 102 so as, in at least one position, not to obstruct the anesthesia vaporizing dials while also not protruding out of the frame 102.

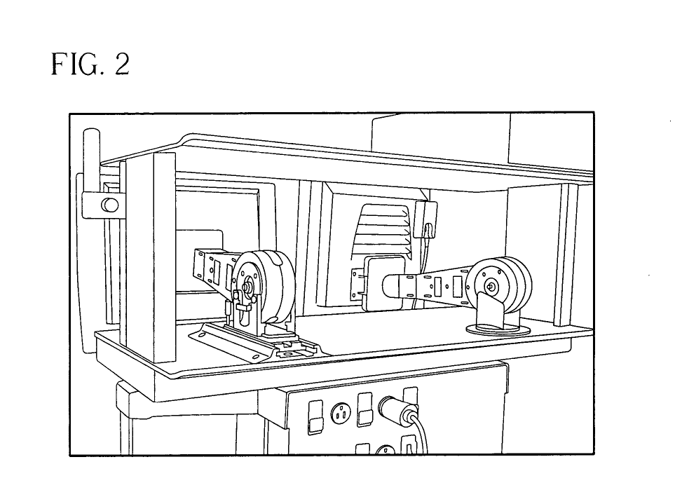

Referring to FIG. 2, the mounting system for the displays, according to one embodiment of the invention, includes a mounting arm with one end adjustably attached to the rear of the monitor and the other end adjustably attached to the frame 102 so as to provide the various degrees of freedom of movement described herein. In one embodiment, the mounting arm is attached to the frame 102 with a horizontal sliding track arranged to provide the fore and aft, or left and right directions of movement.

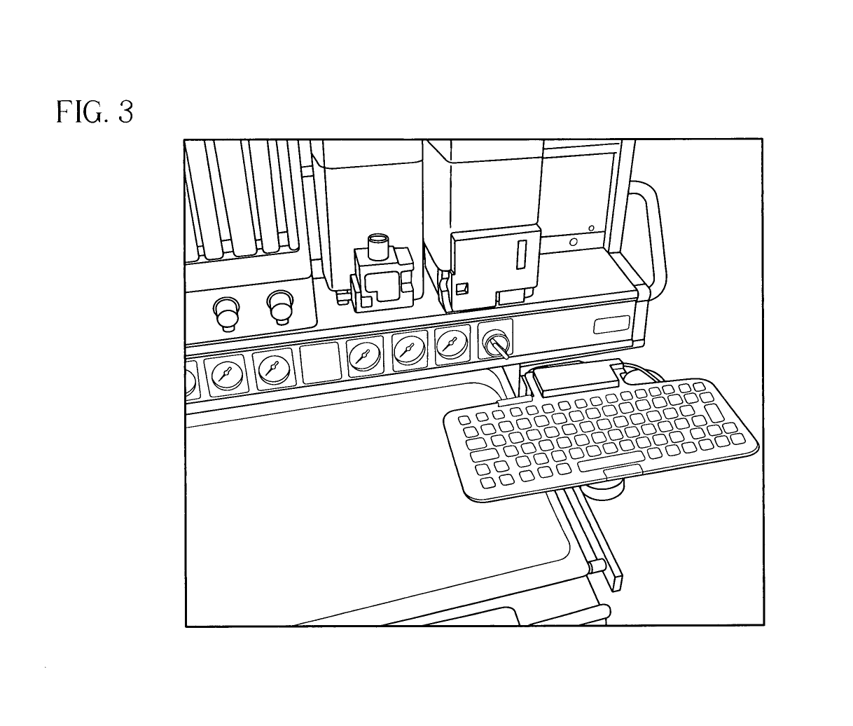

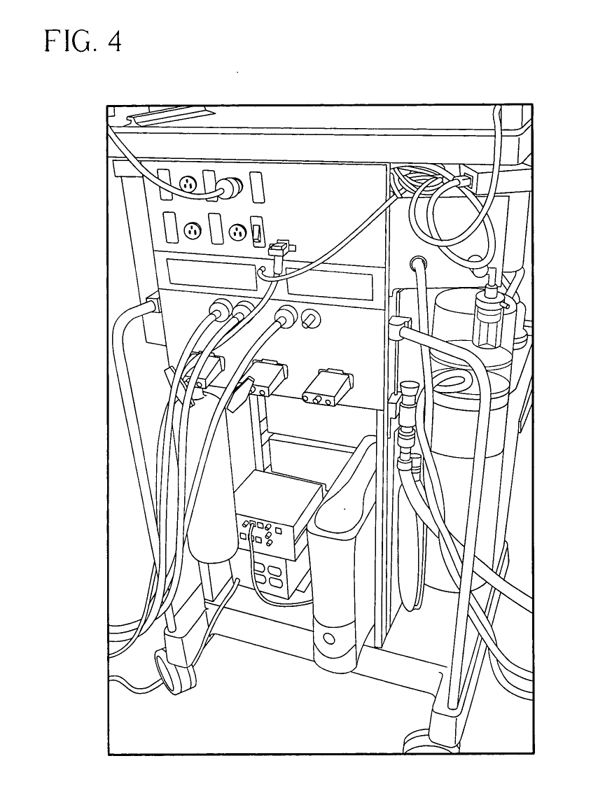

Referring to FIG. 3, the operating room compatible keyboard 106 is, according to one embodiment of the invention, is adjustably attached to the frame 102. The keyboard 106 may similarly be mounted to move in the various directions, e.g., up and down, left and right, back-front, and further mounted to tilt along either or all of the X, Y, and Z axes, to allow the keyboard 106 to fold out of the way toward the side of the frame 102 so that the working part of the keyboard 106, e.g., the keys, is partially or wholly out of the operators field of view. Referring to FIG. 4, a computing device with patient record keeping software associated therewith may beneficially be concealed in the back of the anesthesia machine to provide the relevant functionality described above.

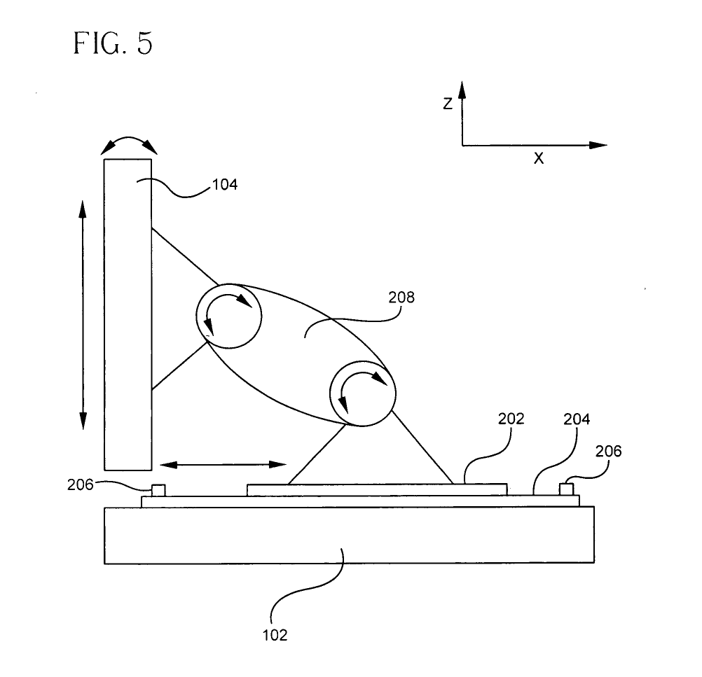

Referring to FIG. 5, the display 104 is adjustable mounted to the frame 102 with a mounting arm 208 with one end adjustably attached to the rear of the display 104 and the other end adjustably attached to the frame 102 with a horizontal sliding track 202, 204 arranged to provide the fore and aft, or left and right directions of movement. The sliding track may also include stops 206 to limit the displays 104 movement. Although the various directions of movement are depicted for illustrative purposes, it is understood that the arm 208 may be attached to the displays 104/frame 102 in a variety of ways that are not shown, and the present invention is thus not limited thereto.

While the foregoing invention has been described in some detail for purposes of clarity and understanding, it will be appreciated by one skilled in the art, from a reading of the disclosure, that various changes in form and detail can be made without departing from the true scope of the invention in the appended claims.

1. An anesthesia machine comprising an anesthesia machine frame and at least one display adjustably attached to the frame, the at least one display mounted in the anesthesia machine so as not to protrude from the frame.

2. The anesthesia machine of claim 1, wherein the at least one display is adopted for patient monitoring.

3. The anesthesia machine of claim 1, wherein the at least one display is adjustably attached to the frame to move in at least one direction selected from the group consisting of: in an in and out direction with respect to the frame, in a left and right direction with respect to the frame, and in an up and down direction with respect to the frame.

4. The anesthesia machine of claim 1, wherein the at least one display is adjustably attached to the frame to allow the display to tilt in at least one direction selected from the group consisting of: in a direction left to right with respect to the frame and in an up-down direction with respect to the frame.

5. The anesthesia machine of claim 1 comprising an operating room compatible keyboard.

6. The anesthesia machine of claim 5, wherein the keyboard is a waterproof keyboard.

7. The anesthesia machine of claim 5, wherein the keyboard is adjustably attached to the anesthesia machine.

8. The anesthesia machine of claim 1, wherein the at least one display is adjustable mounted to the frame with a mounting arm having one end adjustably attached to the display and another end adjustably attached to the frame with a horizontal sliding track arranged to provide movement in at least one of: fore and aft, and left and right movement.

9. The anesthesia machine of claim 1, wherein the at least one display provides an interface for automated record keeping.

10. The device of claim 9, comprising at least one computing device with programs associated therewith for capturing at least one of patient preoperative data and operative data.
